(19) United States
(12) Patent Application Publication
(10) Pub. No.: US 2026/0102611 A1
(43) Pub. Date: Apr. 16, 2026

(54) ORAL APPLIANCE WITH STIMULATOR

(71) Applicant: ACHAEMENID, LLC, Boston, MA (US)

(72) Inventors: Reza Radmand, Boston, MA (US); Yitzhak Mendelson, Worcester, MA (US)

(21) Appl. No.: 19/114,700

(22) PCT Filed: Sep. 25, 2023
(86) PCT No.: PCT/US2023/075048

§ 371 Date: Mar. 24, 2025

Related U.S. Application Data
(60) Provisional application No. 63/409,386, filed on Sep. 23, 2022.
(60) Provisional application No. 63/411,766, filed on Sep. 30, 2022.
(60) Provisional application No. 63/497,605, filed on Apr. 21, 2023.

(51) Int. Cl. A61N 1/36; A61N 1/05

(52) U.S. Cl. CPC A61N 1/36; A61N 1/0548

(57) ABSTRACT

An oral appliance for a user may include a body and a stimulator. The stimulator may include an electrode arranged on or embedded within the body. The stimulator may be in contact with a muscle of a tongue of the user. The stimulator may be configured to stimulate the muscle of the tongue with a stimulation signal to produce contraction in the muscle of the tongue.

### CROSS-REFERENCE TO RELATED APPLICATIONS

This application claims the benefit of and priority to U.S. Provisional Patent Application No. 63/497,605 filed Apr. 21, 2023, U.S. Provisional Patent Application No. 63/411,766 filed Sep. 30, 2022, and U.S. Provisional Patent Application No. 63/409,386 filed Sep. 23, 2022. Each of these applications are incorporated herein by reference in its entirety.

### FIELD OF DISCLOSURE

This disclosure relates generally to oral appliances and more particularly to or appliances having a stimulator.

### BACKGROUND

Sleep apnea is a common medical condition during which a person experiences one or more pauses in breathing and/or shallow breaths during sleep. While there are several types of sleep apnea, the most common type is obstructive sleep apnea. In this medical condition, one or more of the person's throat muscles relax during sleep causing surrounding tissues in the posterior portions of the mouth, nose and throat to collapse, thereby creating a pharyngeal obstruction that can block the upper airway. Persons suffering from obstructive sleep apnea have inadequate oxygen exchange during sleep, which can lead to daytime fatigue, lack of concentration and mood changes. Left untreated, obstructive sleep apnea can have a significant impact on a person's health, often leading to cardiovascular, stroke and metabolic disorders.

### BRIEF SUMMARY

Apparatuses, processes, machines, and articles of manufacture for, or associated with, oral appliances with a stimulator. It will be appreciated that the embodiments may be combined in any number of ways without departing from the scope of this disclosure.

Embodiments may include an oral appliance for a user, comprising: a body; and a stimulator including an electrode arranged on or embedded within the body so that it is in contact with a portion of a tongue of a user, wherein the stimulator is configured to stimulate a muscle of the tongue with a stimulation signal to produce contraction in the muscle of the tongue.

Embodiments may include an oral appliance comprising: a transducer configured to generate data based on one or more physiological parameters; processing circuitry configured to detect a pattern in the data that is indicative of sleep-disordered breathing; and a stimulator configured to generate a stimulation signal including a plurality of bipolar pulses in response to detection of the pattern in the data.

Embodiments may include a computer-implemented method comprising: monitoring breathing data of a user, the breathing data generated by a transducer; detecting a pattern in the breathing data that indicates sleep-disordered breathing; and generating a stimulation signal to stimulate a muscle of a tongue of the user in response to detection of the pattern.

Other apparatuses, processes, machines, and articles of manufacture are also described hereby, which may be combined in any number of ways, such as with the embodiments of the brief summary, without departing from the scope of this disclosure.

### BRIEF DESCRIPTION OF THE SEVERAL VIEWS OF THE DRAWINGS

A more particular description will be rendered by reference to exemplary embodiments that are illustrated in the accompanying figures. Understanding that these drawings depict exemplary embodiments and do not limit the scope of this disclosure, the exemplary embodiments will be described and explained with additional specificity and detail through the use of the accompanying drawings. To easily identify the discussion of any particular element or act, the most significant digit or digits in a reference number refer to the figure number in which that element is first introduced.

FIG. 1 illustrates a block diagram of an oral appliance including a stimulator according to some embodiments.

FIG. 2 illustrates an exemplary removable oral appliance including a stimulator according to some embodiments.

FIG. 3 illustrates exemplary tongue muscles for stimulation according to some embodiments.

FIG. 4A illustrates an oral appliance positioned in a mouth when stimulation is off according to some embodiments.

FIG. 4B illustrates an oral appliance positioned in a mouth when stimulation is on according to some embodiments.

FIG. 5A illustrates an oral appliance positioned in a mouth when stimulation is off according to some embodiments.

FIG. 5B illustrates an oral appliance positioned in a mouth when stimulation is on according to some embodiments.

FIG. 6 illustrates various aspects of an exemplary stimulation signal according to some embodiments.

FIG. 7 illustrates various aspects of an exemplary stimulation signal according to some embodiments.

FIG. 8 illustrates various aspects of an exemplary stimulation signal according to some embodiments.

FIG. 9 illustrates various aspects of an exemplary stimulation signal according to some embodiments.

FIG. 10 illustrates various aspects of an exemplary stimulation signal according to some embodiments.

FIG. 11 illustrates various aspects of an exemplary stimulation signal according to some embodiments.

FIG. 12 illustrates various aspects of an exemplary stimulation signal according to some embodiments.

FIG. 13 illustrates a block diagram of an oral appliance including a mandible appliance with a stimulator and a maxilla appliance with a transducer according to some embodiments.

FIG. 14 illustrates an exemplary removable maxilla appliance including a sound measuring device according to some embodiments.

### DETAILED DESCRIPTION

Reference will now be made in detail to various exemplary embodiments. Each example is provided by way of explanation and is not meant as a limitation and does not constitute a definition of all possible embodiments. It is understood that reference to a particular “exemplary embodiment” of, e.g., a structure, assembly, component, configuration, method, etc. includes exemplary embodiments of, e.g., the associated features, subcomponents, method steps, etc. forming a part of the “exemplary embodiment”.

For purposes of this disclosure, the phrases “devices,” “systems,” and “methods” may be used either individually or in any combination referring without limitation to disclosed components, grouping, arrangements, steps, functions, or processes. Reference in the specification to “one embodiment” or “an embodiment” means that a particular feature, structure, or characteristic described in connection with the embodiment can be included in at least one embodiment of the disclosure. The appearances of the phrase “in one embodiment” in various places in the specification do not necessarily all refer to the same embodiment.

Embodiments of the disclosure generally relate to devices and methods for treating obstructive sleep apnea, as well as a device for providing electrical stimulation to a user's tongue to inhibit and/or limit snoring that may be caused by obstructive sleep apnea. Such devices provide particular utility in providing electrical stimulation to the user's tongue in such a manner that the stimulation does not awaken the user during sleep. The oral appliance contemplated includes a mouthpiece that is configured to receive at least temporary, permanent and/or artificial lower dentition of the user.

Existing techniques for treatment of obstructive sleep apnea include both surgical and nonsurgical devices. A popular surgical procedure is uvulopalatopharyngoplasty, which may be performed for patients who have anatomical abnormalities that cause their obstructive sleep apnea and/or make them less likely to tolerate nonsurgical devices. Uvulopalatopharyngoplasty may be a complicated surgery, during which a portion of the soft palate is removed in an effort to prevent closure of the airway by excess tissue during sleep. A disadvantage of this procedure, however, is that the operation is often expensive and may damage throat muscles necessary for swallowing and/or cause other undesirable disorders, such as, nasal regurgitation and numbness of the lower front teeth.

To reduce this risk, various nonsurgical approaches have been employed. One such nonsurgical approach includes using standardized oral appliances to incrementally advance and/or protrude the mandible (lower jaw) relative to the maxilla (upper jaw). These standardized oral appliances, commonly referred to as a mandibular advancement device, (“MAD”), typically include upper and lower dental trays, whereby the lower dental tray is designed to advance the mandible, and hence, move the tongue forward to increase the space in the posterior part of the throat and the oropharynx, which in turn may serve to increase the flow of air during sleep. The distance (degree of advancement) required to protrude and/or reposition the mandible may be, at least in part, dependent on the severity of the individual's obstructive sleep apnea, as well as psychological variables among the users. A disadvantage of using these standard oral appliances is that they may not sufficiently provide for and/or address individualized anatomical variances, such as difference in dental arches, dentition alignment and/or jaw flexibility. Another disadvantage is that in instances where the degree of advancement is excessive, the appliance may lead to long-term temporomandibular joint (“TMJ”) disorders, muscular aggravation, dentition discomfort and/or myofascial disorders. As a result, use of these standard appliances has an approximate compliance rate of 75% over a 2-year period. For a detailed study of compliance with use of MAD, see Non-CPAP therapies in obstructive sleep apnoea: mandibular advancement device therapy, see Eur Respir J 2012; 39:1241-1247, which is incorporated by reference in its entirety. Thus, such oral appliances may not treat obstructive sleep apnea in a manner that prevents and/or limits impacts on a person's health.

Existing methods of treating obstructive sleep apnea include the administration of positive air pressure via a continuous positive airway pressure (“CPAP”) machine. The CPAP machine is often assembled for use in combination with various face or nasal masks and may provide continuously pressurized and/or forced air during the person's sleep. A disadvantage of this assembly is that it may cause nasal and/or oral mucosal dryness due to the continuously forced air and may also cause claustrophobia due to the presence of a mask on the patient's face. As a result, use of these assemblies has an approximate compliance rate of 50% over a 5-year period. For a detailed study of compliance with use of CPAP machines, see Long-term compliance with continuous positive airway pressure in patients with obstructive sleep apnea, Can Respir J. 2008 October; 15(7): 365-369, which is incorporated by reference in its entirety. Another disadvantage is that standard masks are not properly adapted for a customized fit for persons with unique and/or variable facial anatomies that may be natural or created by loss of muscle tone secondary to facial paralysis and/or stroke. Ill-fitting masks may lead to leakage of air and/or inadequate air intake. In addition, the masks used with CPAP machines have been found to be a breeding ground for bacteria and fungi. Despite routine washing and cleaning measures, the bacteria and fungi on these masks can grow exponentially, and lead to infections, such as pneumonia, in the airways of persons who use them. Moreover, such assemblies may not sufficiently treat obstructive sleep apnea and may fail to promote patient compliance with the treatment method.

The aforementioned treatment techniques may not provide sufficient treatment of obstructive sleep apnea, may cause and/or promote other negative health situations for the user and may not foster compliance with treatment methods.

In view of the disadvantages associated with currently available methods and devices for treating obstructive sleep apnea, there is a need for a device and method that treats obstructive sleep apnea while storing patient behavior and/or medical data relating to a user's breathing pattern, snoring pattern and/or clenching/grinding behaviors, that can assist medical providers in the design, improvement and/or modification of specialized treatment measures for individual patients. Further, there is a need for a device and method that treats obstructive sleep apnea in a single removable oral appliance and prevents and/or limits long-term TMJ disorders, muscular aggravation and/or myofascial disorders that may occur with continued use of currently available appliances.

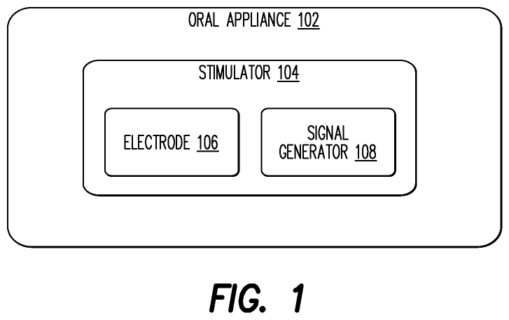

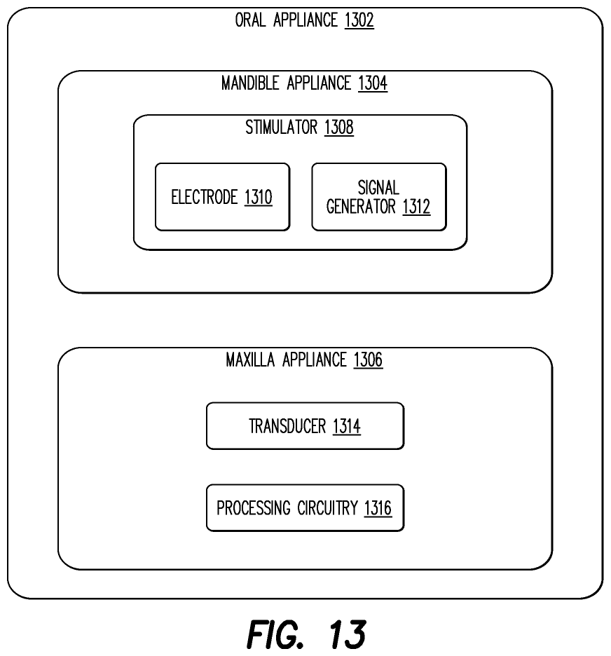

FIG. 1 illustrates a block diagram of an oral appliance 102 including a stimulator 104 according to some embodiments. The stimulator 104 of the oral appliance 102 may include an electrode 106 and a signal generator 108. In various embodiments, the electrode 106 may be positioned to deliver stimulation signals generated by the signal generator 108 to a tongue of a user. The stimulation signals may be configured to deliver electrical impulses to a tongue of a user to produce contraction in one or more tongue muscles of the user response to an event, such as sleep-disordered breathing, relaxation of a tongue muscle, or a sleep apnea event. For example, as described in more detail below, such as with respect to FIG. 13, the stimulator 104 may be utilized to cause a tongue of a user to contract in response to detection of a breathing pattern indicative of an event comprising sleep-disordered breathing. Embodiments are not limited in this context.

In many embodiments, the stimulator 104 may include at least one electrode 106 configured to provide at least intermittent stimulation to one or more muscles of the tongue of a user, such as one or more of a super longitudinal, inferior longitudinal, palatoglossus, styloglossus, and genioglossus muscles of the tongue until the event has ceased. In some embodiments, the one or more electrodes 106 may include mesh electrodes positioned in contact with a tongue of a user. In some such embodiments, the mesh electrodes may extend along the lateral border of a tongue of a user. For example, the mesh electrodes may be in contact with a ventral-lateral surface at a mid to anterior genioglossus muscle of the tongue or a ventral-lateral at a mid to anterior genioglossus surface of the tongue.

The stimulator 104 may be activated if the user's oxygen saturation levels are below a certain level, if the user is grinding teeth, and/or if inspiration and expiration levels of the user has decreased below a threshold. Stimulation of the tongue muscle may facilitate an increase in respiratory flow to the user, thereby increasing the availability of oxygen to the user and the increase of oxygen saturation levels of hemoglobin. It is contemplated that the stimulator 104 will provide gentle electrical stimulation to the genioglossus muscle of the tongue.

In one embodiment, the stimulator 104 is configured to provide constant stimulation to one or more muscles of the tongue. Alternatively, the stimulator 104 may provide variant stimulation to one or more muscles of the tongue. In some embodiments, the variant stimulation may increasingly stimulate the genioglossus muscle of the tongue until the sleep apnea event has ceased.

One method of using the oral appliance 102 may include inserting the oral appliance 102 in the oral cavity of a user. The tongue stimulating oral appliance may be inserted into the patient's mouth when they are awake or once they are in the operating room, either during while they are still awake or once they are under drug-induced sleep, such as part of a drug-induce sleep endoscopy (DISE). If the patient is under drug-induced sleep, transcutaneous electrical stimulation of the tongue can commence after the laryngoscope has been inserted into the airway. The tongue may be stimulated at the start of inspiration, likely during a period of flow limitation to observe improvements in flow with electrical stimulation. If the patient becomes aroused from sleep upon electrical stimulation of the tongue, the tongue stimulation protocol may be aborted. Arousal from sleep during DISE can be observed in the airflow data as an acute increase in airflow for several consecutive breaths (similar to appearance of an arousal from sleep on regular sleep study).

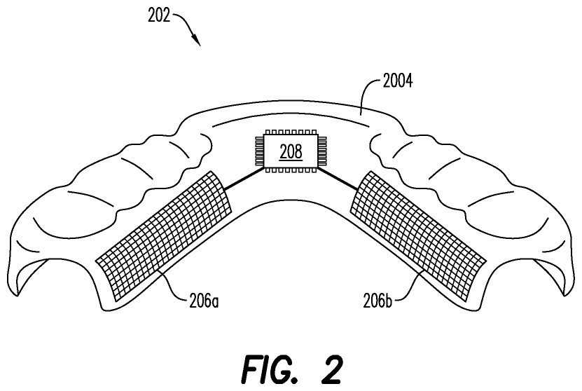

FIG. 2 illustrates a removable oral appliance 202 including a body 204 and a stimulator according to some embodiments. In the illustrated embodiment, the stimulator includes mesh electrodes 206a, 206b (collectively referred to as mesh electrodes 206) and signal generator 208. The body 204 of the oral appliance 202 may include a mouthpiece made of dental acrylic or plastic material. According to an aspect, the body 204 is customized to be receivably positioned and/or secured on the mandible of the user. According to an aspect, the body 204 is customized to receive the lower dentition of the user. In any event the body 204 may be customized such that it provides a comfortable fit that enhances the user's comfort and reinforces the user's likelihood of repeated wear of the mouthpiece, i.e., the user's compliance rate. Embodiments are not limited in this context.

The mesh electrodes 206 and the signal generator 208 may be embedded in a wall and/or disposed on a surface of the body 204 of the oral appliance 202. According to an aspect, the one or more mesh electrodes may extend about 3 cm along the lateral border of the tongue. In the illustrated embodiment, a pair of mesh electrodes 206 are be provided, with each electrode positioned in the inner wall (lingual side) of the oral appliance, so the electrodes are bilaterally arranged. According to an aspect, the electrodes are disposed on the oral appliance so that they will engage the ventral-lateral at the mid to anterior genioglossus surface of the user's tongue. For example, the first mesh electrode 206a may be positioned on the left side of the tongue, while a second mesh electrode 206b (spaced apart from the first electrode) is positioned on the right side of the tongue.

The two mesh electrodes 206 may be independently connected to the signal generator 208 with a 0.6 mm diameter electric wire that may also be embedded in the wall of the body 204 of the oral appliance 202. In some embodiments, the signal generator 208 may be external to the mouth of a user and connected via wires. Although a wired configuration is illustrated and described, in some embodiments, one or more wires may be replaced with wireless connections. Further, in various embodiments, the signal generator 208 may wirelessly communicate with an external computing device, such as for configuring operational parameters of the signal generator 208.

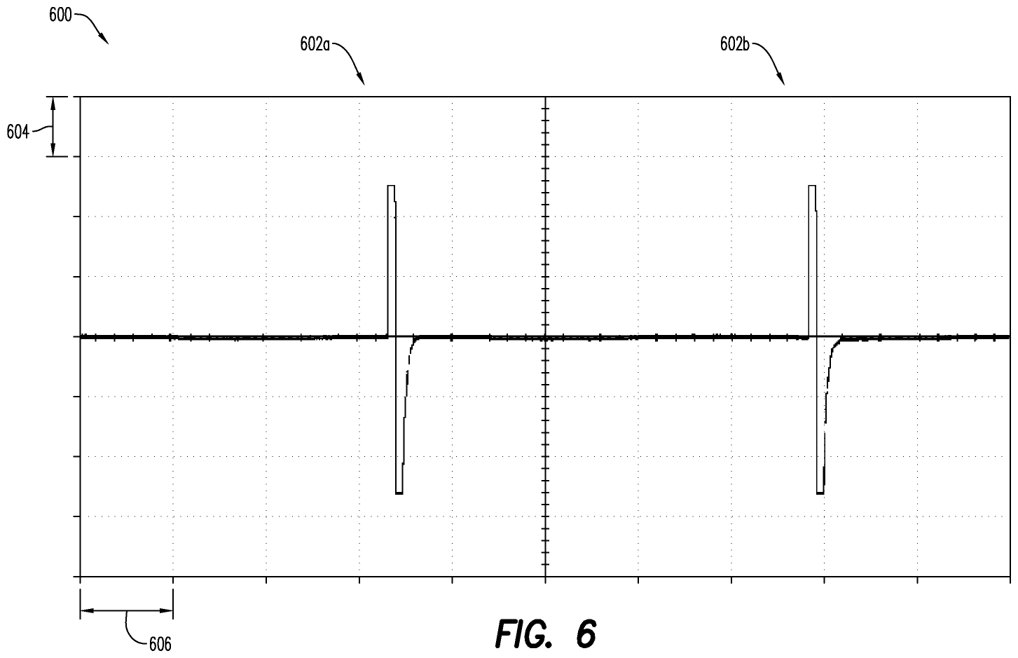

It is contemplated that the electrodes may generate electrical impulses in response to stimulation signals generated by the signal generator 208. For example, the electrical impulses may have a biphasic width of about 100 micro-seconds, at a rate of approximately about 10 Hertz (Hz) to about 15 Hz, in bursts of about 2 seconds to about 6 seconds. The stimulation signals are described in more detail below, such as with respect to FIGS. 6-12. In some embodiments, the impulses may be administered in a controlled manner at the onset of air flow reduction at the beginning of inspiration under DISE. The intensity of stimulation may be limited to levels that may be well tolerated by a user during wakefulness, such as between 0 and 30 volts (e.g., 2V, 3.7V, 5V, 10V, 15V, etc.).

In various embodiments, the signal generator 208 may include, or provide, functionality that is the same or similar to a transcutaneous electrical nerve stimulator (TENS unit/device), such as a portable ProM-300 TENS device manufactured by ProMed Specialties, Huntingdon, PA. Such a TENS unit is a device designed to send comfortable impulses through the skin to stimulate the nerve (or nerves) in the treatment area for the purpose of relieving pain. However, in embodiments described hereby, the TENS device may be utilized in an atypical manner to electrically stimulate muscles to cause a muscle contraction in a tongue of a user. It is contemplated that the TENS device can comfortably stimulate the tongue muscle to produce contraction. Furthermore, protocol stimulating the tongue using needle electrodes inserted into the genioglossus muscle and attached to a neuromuscular stimulator have been developed (Dynex III, Medtronic Inc. Minneapolis, MN, USA) that found that the tongue muscle could be electrically stimulated safely during drug-induced sleep to increase airflow, without causing arousal from sleep. Taken together, it is contemplated by this disclosure that tongue stimulation using the TENS device or a similar device, is not associated with any significant risks to a user. Specifically, given that the stimulation can be tolerated during wake, discomfort while under drug-induced sleep, for example, is unlikely.

According to an aspect, the transcutaneous electrical impulses are applied to the surface of the tongue using biocompatible mesh securely attached to the appliance using a biocompatible adhesive, such as Dermabond®. In various embodiments, the mesh may comprise one or more of stainless steel, gold, silver, and silver chloride. The oral appliance 202 may be made from a biocompatible dental plastic acrylic that is used clinically to make dental appliances (such as Drusoft® pro), which has a CE label and therefore fulfills the general requirement of medical device guidelines.

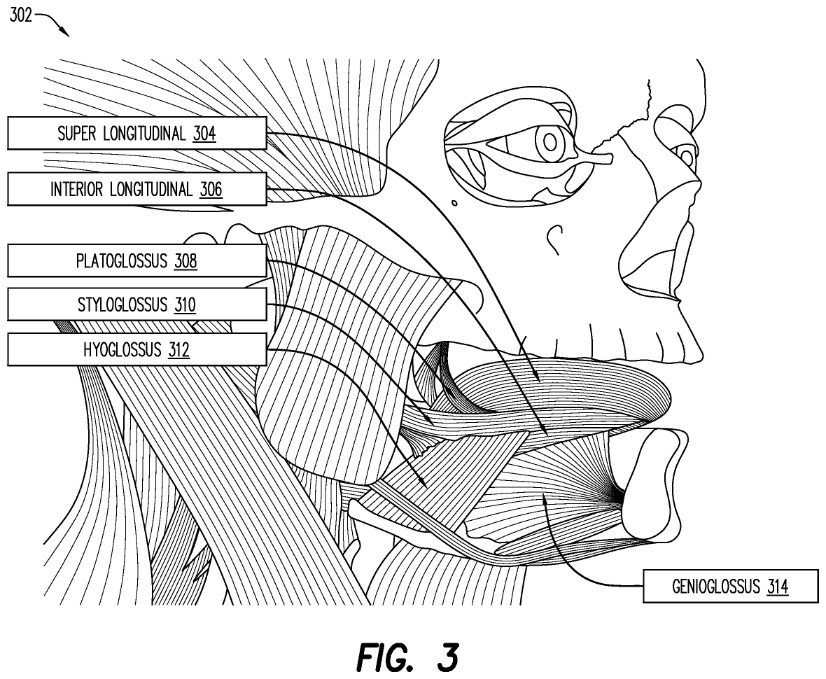

FIG. 3 illustrates tongue muscles 302 for stimulation according to some embodiments. In the illustrated embodiment, the tongue muscles 302 include the super longitudinal 304, the inferior longitudinal 306, the palatoglossus 308, the styloglossus 310, the hyoglossus 312, and the hyoglossus 312. In various embodiments, one or more of the tongue muscles 302 may be stimulated to cause movement of the tongue to unobstruct the posterior pharyngeal space. In various such embodiments, this allows for a larger volume of air to be exchanged and reduces obstructed sleep apnea episodes. For example, the combination of superior and inferior longitudinal muscles 304, 306, either by themselves or in concert with one or more of the styloglossus 310, palatoglossus 308 and hyoglossus 312, may be stimulated. Embodiments are not limited in this context.

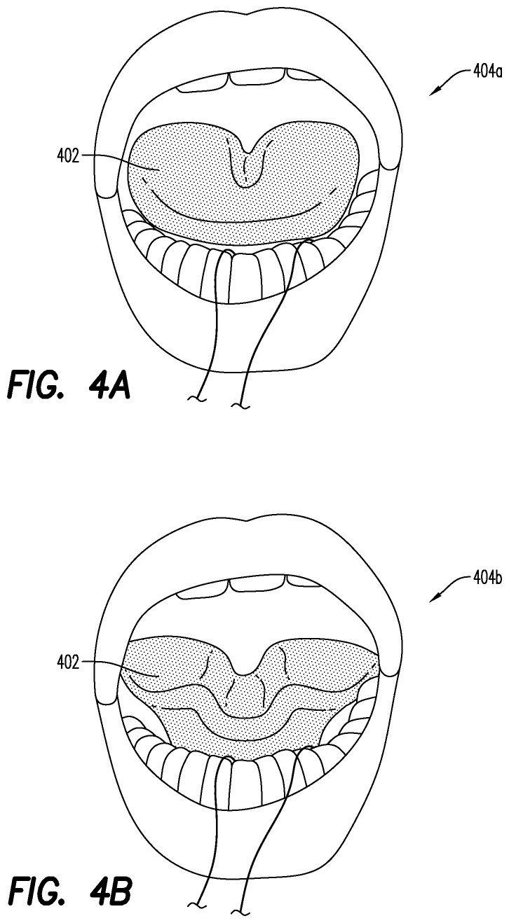

FIG. 4A illustrates an oral appliance positioned in a mouth when stimulation is off according to some embodiments. As seen in FIG. 4A, the tongue 402 is in a relaxed, un-contracted state 404a. Embodiments are not limited in this context.

FIG. 4B illustrates an oral appliance positioned in a mouth when stimulation is on according to some embodiments. The stimulation provided by be constant or variant. It is contemplated that the constant or variant stimulation may be a gentle stimulation that does not disturb and/or awaken the user during sleep. According to an aspect, the constant or variant stimulation is gentle enough so that the user does not recognize it when wearing it when the user is at least slightly awake. As seen in FIG. 4B, the tongue 402 of the user is now in a contracted state 404b, which pulls the tongue 402 away from the back of the throat of the user so the user can breathe. Embodiments are not limited in this context.

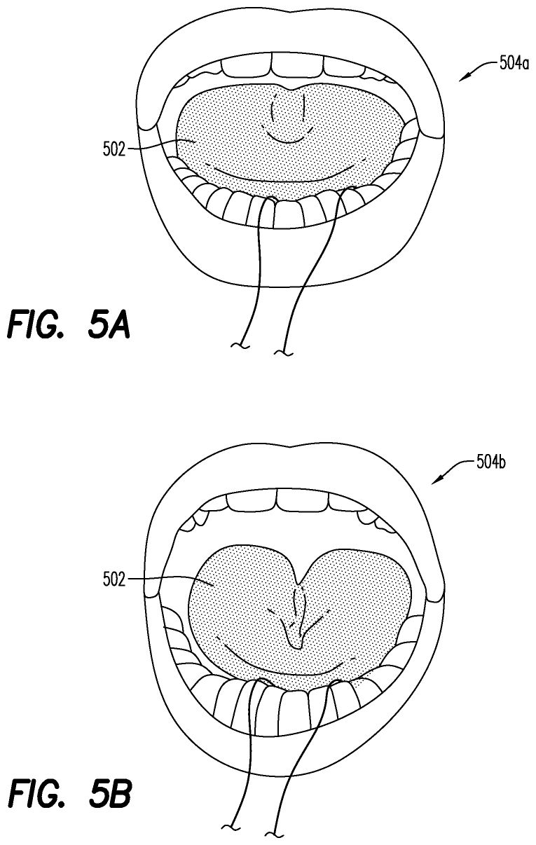

FIG. 5A illustrates an oral appliance positioned in a mouth when stimulation is off according to some embodiments. Similar to FIG. 4A, as seen in FIG. 5A, the tongue 502 is in a relaxed, un-contracted state 504a. Embodiments are not limited in this context.

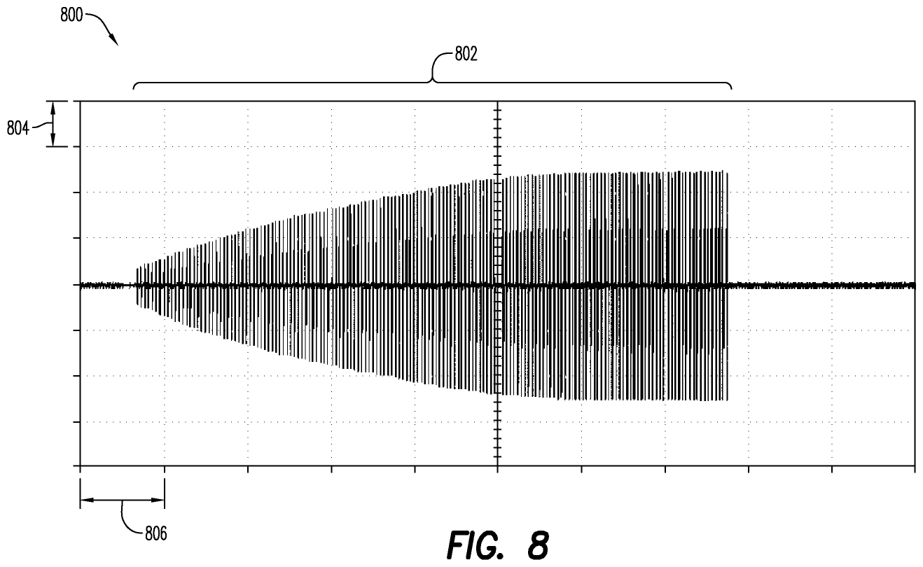

FIG. 5B illustrates an oral appliance positioned in a mouth when stimulation is on according to some embodiments. Similar to FIG. 4B, as seen in FIG. 5B, the tongue 502 of the user is now in a contracted state 504b, which pulls the tongue 502 away from the back of the throat of the user so the user can breathe. The differences in the contraction of the tongues shown in FIGS. 4B and 5B may be the result of using different stimulation signals. For example, the contracted state 404b of FIG. 4B may result from a ramped stimulation signal (see e.g., FIG. 8) while the contracted state 504b of FIG. 5B may result from a constant stimulation signal. In various embodiments, contraction of the tongue may cause the tongue to move anteriorly and away from the back of the throat (protrusion). In some embodiments, the contraction may additionally, or alternatively, result in the reduction of the size of the muscles which then occupies less space laterally and/or antero-posteriorly. Embodiments are not limited in this context.

FIG. 6 illustrates various aspects of a stimulation signal 600 according to some embodiments. The stimulation signal 600 includes a first bipolar pulse 602a and a second bipolar pulse 602b and is illustrated on a chart having an x-axis corresponding to time and a y-axis corresponding to voltage. The chart includes a y-axis scale 604 of 2 volts and an x-axis scale 606 of 5 milliseconds (ms), resulting in the y-axis of the chart extending from −8V to +8V and the x-axis of the chart extending from 0 ms to 50 ms. Accordingly, the first and second bipolar pulses 602a, 602b of the stimulation signal 600 each include a positive portion with approximately a 5V amplitude (referred to as the intensity) occurring for approximately 2 ms and a negative portion with approximately a −5V intensity occurring for approximately 2 ms. Additionally, a gap of approximately 20 ms is between the first bipolar pulse 602a and the second bipolar pulse 602b. Embodiments are not limited in this context.

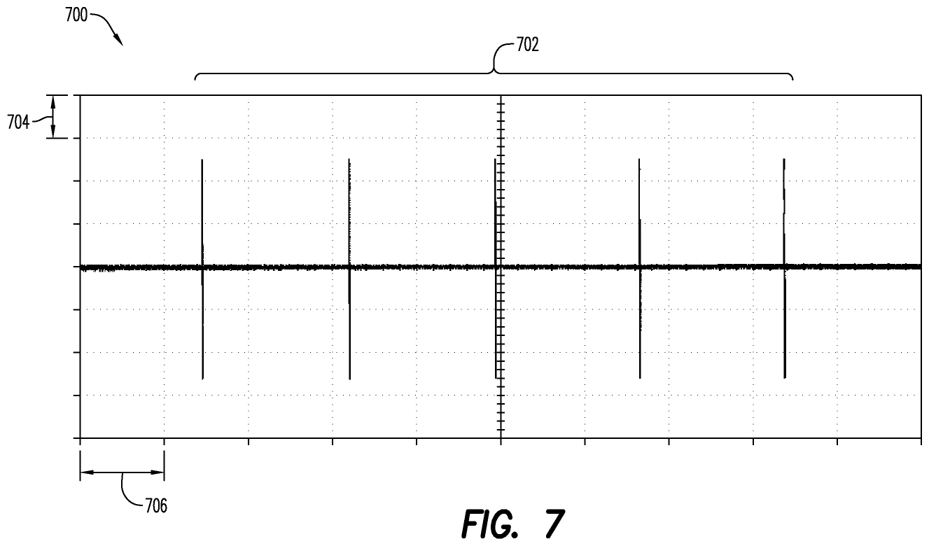

Generally, the area under the pulses may correspond to the charge delivered to the user. In many embodiments, the total charge delivered to a patient may be as close to zero as possible, such as for user comfort. Accordingly, the area under the positive portion of a bipolar pulse may be approximately equal to the area under the negative portion of a bipolar pulse. Although it may not be apparent, such as due to scaling, sampling, and/or rendering factors, the stimulation signals described with respect to FIGS. 7-12 are composed of a plurality of bipolar pulses having the same duration as bipolar pulses 602a, bipolar pulse 602b. However, as described in more detail below, the amplitudes of the bipolar pulses may be varied.

FIG. 7 illustrates various aspects of a stimulation signal 700 according to some embodiments. The stimulation signal 700 includes a pulse train 702 and is illustrated on a chart having an x-axis corresponding to time and a y-axis corresponding to voltage. The chart includes a y-axis scale 704 of 2 volts and an x-axis scale 706 of 500 milliseconds (ms), resulting in the y-axis of the chart extending from −8V to +8V and the x-axis of the chart extending from 0 to 5 seconds. The pulse train 702 has a duration of approximately 3.5 seconds and includes five bipolar pulses that are the same or similar to bipolar pulse 602a and bipolar pulse 602b having a +/−5 volt intensity. Further, the pulse train 702 includes a gap of approximately 0.75 seconds between each pulse. In many embodiments, a pulse train may include bipolar pulses with a variety of amplitudes. In many such embodiments, the pulse train may include at least one of a ramp-up portion, a level portion, or a ramp-down portion in which the amplitudes of the bipolar pulses of the pulse train may vary (e.g., during the ramp-up portion or the ramp-down portion). Embodiments are not limited in this context.

FIG. 8 illustrates various aspects of a stimulation signal 800 according to some embodiments. The stimulation signal 800 includes a pulse train 802 and is illustrated on a chart having an x-axis corresponding to time and a y-axis corresponding to voltage. The chart includes a y-axis scale 804 of 2 volts and an x-axis scale 806 of 500 milliseconds (ms), resulting in the y-axis of the chart extending from −8V to +8V and the x-axis of the chart extending from 0 to 5 seconds. The pulse train 802 has a duration of approximately 3.5 seconds and includes a plurality of bipolar pulses that are the same or similar to bipolar pulse 602a and bipolar pulse 602b having approximately a 20 ms gap between each pulse. However, the bipolar pulses of pulse train 802 ramp from 0 to +/−5V in a curved manner (as opposed to linear) and then level off at +/−5 volts. The ramp from 0 to +/−5V may occur in approximately the first 2 seconds of the pulse train 802. More generally, the ramp can be from a first voltage to a second voltage over at least a portion of the duration of the pulse train. Embodiments are not limited in this context.

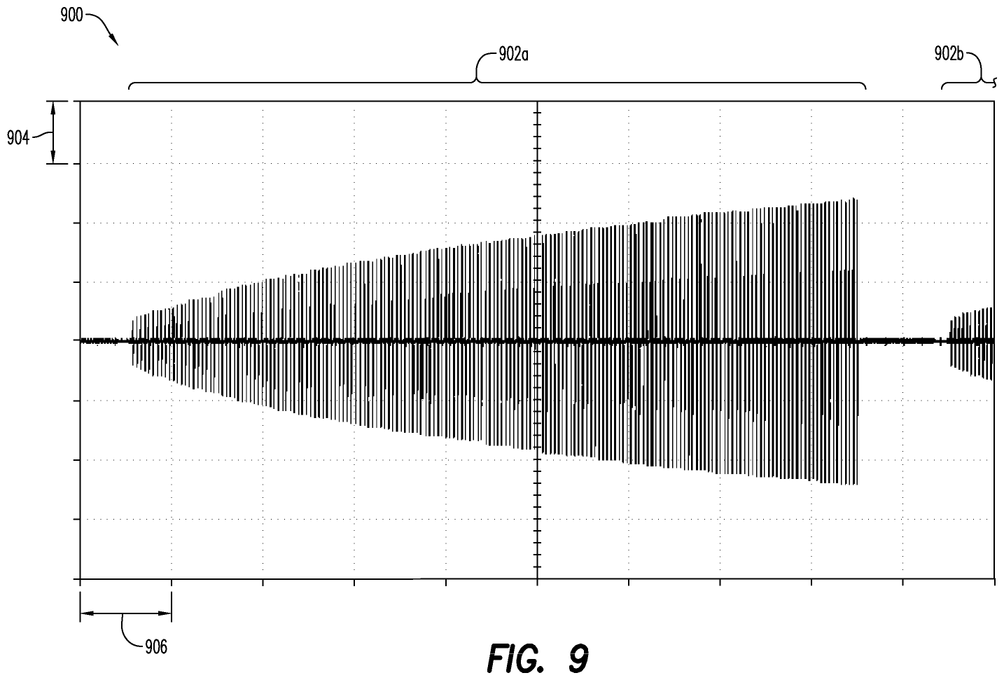

FIG. 9 illustrates various aspects of a stimulation signal 900 according to some embodiments. The stimulation signal 900 includes first and second pulse train pulse trains 902a, 902b and is illustrated on a chart having an x-axis corresponding to time and a y-axis corresponding to voltage. The chart includes a y-axis scale 904 of 2 volts and an x-axis scale 906 of 500 milliseconds (ms), resulting in the y-axis of the chart extending from-8V to +8V and the x-axis of the chart extending from 0 to 5 seconds. The first pulse train 902a has a duration of approximately 4 seconds and includes a plurality of bipolar pulses that are the same or similar to bipolar pulse 602a and bipolar pulse 602b having approximately a 20 ms gap between each pulse. However, the bipolar pulses of pulse train 902a ramp from 0 to +/−5V. The ramp of pulse train 902a may occur in a linear manner (as opposed to curved). Only a portion of the pulse train 902b is illustrated, however the second pulse train 902b may be the same as the first pulse train 902a. The gap between the first and second pulse trains 902a, 902b may be approximately 0.5 seconds. In various embodiments, a stimulation signal may include a plurality of pulse trains that occur over a predetermined amount of time and/or that occur until an event indicative sleep-disordered breathing resides. Gaps between the pulse trains, or in between individual pulses, may allow the muscle to relax. This relaxation can reduce or eliminate muscle fatigue and/or pain resulting from the stimulation. In some embodiments, the gap can be 3-4 seconds. Embodiments are not limited in this context.

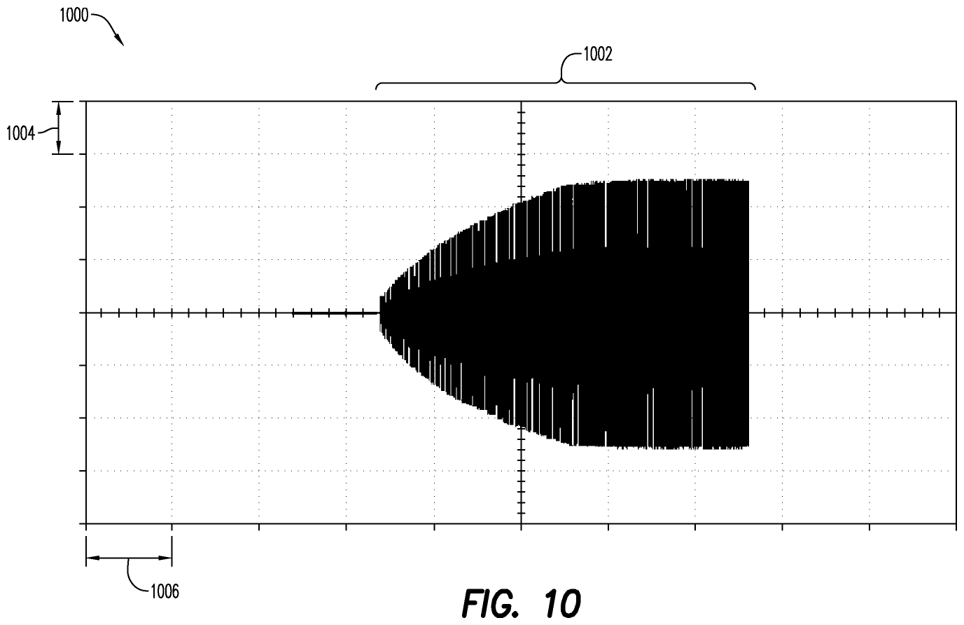

FIG. 10 illustrates various aspects of a stimulation signal 1000 according to some embodiments. The stimulation signal 1000 includes pulse train 1002 and is illustrated on a chart having an x-axis corresponding to time and a y-axis corresponding to voltage. The chart includes a y-axis scale 1004 of 2 volts and an x-axis scale 1006 of 2 seconds, resulting in the y-axis of the chart extending from −8V to +8V and the x-axis of the chart extending from 0 to 20 seconds. The pulse train 1002 has a duration of approximately 8 seconds and includes a plurality of bipolar pulses that are the same or similar to bipolar pulse 602a and bipolar pulse 602b having approximately a 20 ms gap between each pulse. However, the bipolar pulses of pulse train 1002 ramp from 0 to +/−5V and then level off at 5. The ramp of pulse train 1002 may occur in a curved manner (as opposed to linear) and then level off at +/−5 volts. The ramp from 0 to +/−5V may occur in approximately the first 4 seconds of the pulse train 1002. Embodiments are not limited in this context.

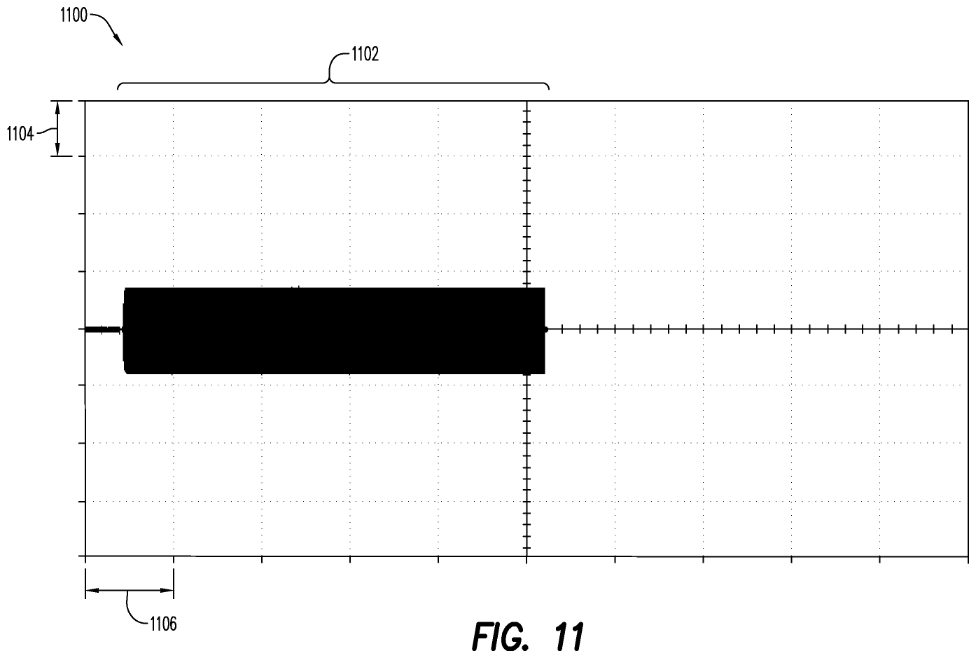

FIG. 11 illustrates various aspects of a stimulation signal 1100 according to some embodiments. The stimulation signal 1100 includes pulse train 1102 and is illustrated on a chart having an x-axis corresponding to time and a y-axis corresponding to voltage. The chart includes a y-axis scale 1104 of 2 volts and an x-axis scale 1106 of 2 seconds, resulting in the y-axis of the chart extending from −8V to +8V and the x-axis of the chart extending from 0 to 20 seconds. The pulse train 1102 has a duration of approximately 9.5 seconds and includes a plurality of bipolar pulses that are the same or similar to bipolar pulse 602a and bipolar pulse 602b having approximately a 20 ms gap between each pulse. However, the bipolar pulses of pulse train 1102 have an intensity of approximately +/−1.8V. Embodiments are not limited in this context.

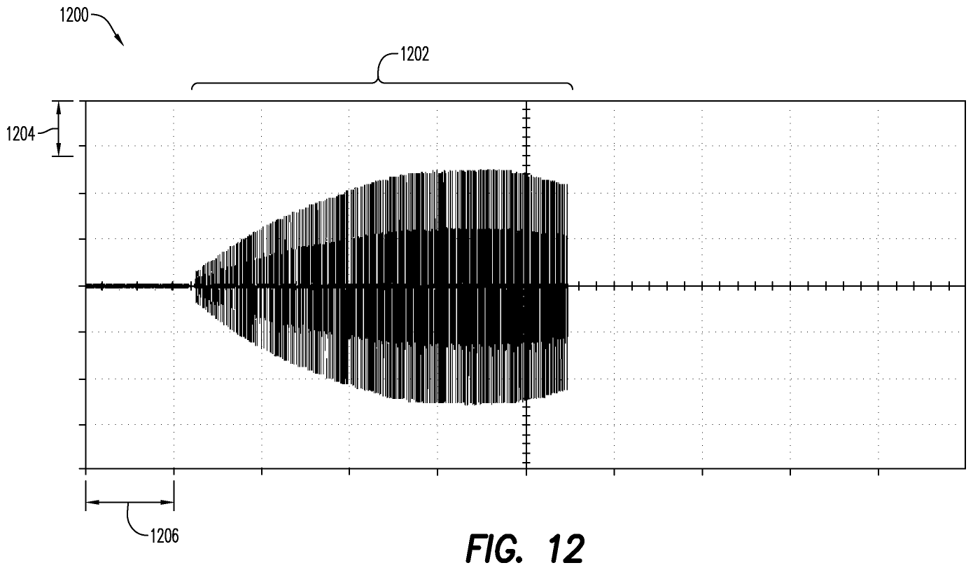

FIG. 12 illustrates various aspects of a stimulation signal 1200 according to some embodiments. The stimulation signal 1200 includes pulse train 1202 and is illustrated on a chart having an x-axis corresponding to time and a y-axis corresponding to voltage. The chart includes a y-axis scale 1204 of 2 volts and an x-axis scale 1206 of 1 second, resulting in the y-axis of the chart extending from −8V to +8V and the x-axis of the chart extending from 0 to 10 seconds. The pulse train 1202 has a duration of approximately 4.1 seconds and includes a plurality of bipolar pulses that are the same or similar to bipolar pulse 602a and bipolar pulse 602b having approximately a 20 ms gap between each pulse. However, the bipolar pulses of pulse train 1202 ramp from 0 to +/−5V, level off at +/−5V, and then ramp from +/−5V to +/−4.2V. The ramp-up of pulse train 1202 may occur in a curved manner (as opposed to linear) in approximately the first 2.75 seconds. The level portion of pulse train 1202 may occur approximately between 2.75 seconds and 3.5 seconds. The ramp-down of pulse train 1202 may occur in a linear manner (as opposed to curved) in approximately the last 0.6 seconds. More generally, the ramp-down can be from a first voltage to a second voltage over at least a portion of the duration of the pulse train. The level portion of the pulse train may occur over at least a portion of the pulse train. Embodiments are not limited in this context.

The stimulation signals described and illustrated with respect to FIGS. 6-12 may be selectively implemented by signal generators to stimulate tongue muscles of users. The specific characteristics of the stimulation signals may be tailored for each user. For example, stimulation signal 800 may be most effective for a first user while stimulation signal 1200 may be most effective for a second user. Further, the intensities, ramp-up (curved or linear), ramp-down (curved or linear), gaps, durations, and the like may be configured for each user and/or reconfigured. For example, users may experience an increase in tolerance to the stimulation signals over time, resulting in their stimulation signal being reconfigured to have a higher intensity (e.g., 10V) and/or duration (e.g., 5 seconds). In another example, the gap between pulse trains may be between 1 and 10 seconds, such as 3 or 4 seconds. The current delivered by the stimulation signals may vary based on the impendence of each user. For example, the tissue of some users may have a higher impedance than the tissue of other users. Accordingly, configuration of the stimulation signal for a user may be influenced by the impedance of the tissue of the user. Additional user characteristics, such as humidity levels in the mouth (e.g., due to dry mouth) may influence the impedance associated with a user.

FIG. 13 illustrates a block diagram of an oral appliance 1302 including a mandible appliance 1304 and a maxilla appliance 1306 according to some embodiments. The mandible appliance 1304 may include a stimulator 1308 with an electrode 1310 and a signal generator 1312. In various embodiments, the mandible appliance 1304 may be the same or similar to oral appliance 102. The maxilla appliance 1306 may include a transducer 1314 and processing circuitry 1316. In various embodiments, the processing circuitry 1316 may monitor data generated by transducer 1314 to detect events indicative of sleep-disordered breathing. In response to detection of the event, the processing circuitry 1316 may cause the stimulator 1308 to stimulate tongue muscles as previously described. When the event subsides, the processing circuitry 1316 may cause stimulator 1308 to cease stimulating the tongue muscles. Embodiments are not limited in this context.

The transducer 1314 may include one or more sensors for generating data corresponding to various physiological parameters that are indicative of sleep-disordered breathing. For example, data generated by the one or more sensors may correspond to one or more of temperature, oxygen levels, inspiration, expiration, tongue position, head position, oxygen saturation, breathing, snoring, clenching/grinding, and the like may be monitored. The processing circuitry 1316 may monitor this data to detect patterns indicative of sleep-disordered breathing and activate/deactivate the signal generator 1312 to generate various stimulation signals in response thereto. For example, breathing data of a user may be monitored to detect a pattern indicative of sleep-disordered breathing.

The processing circuitry 1316 may include a processor and memory. The memory may store instructions that when executed by the processor cause the processor to perform one or more operations or functions described herein, such as detecting patterns indicative of sleep-disordered breathing and activating/deactivating the signal generator 1312. Further, the memory may store data generated by transducer 1314. For example, the processor may include one or more of include one or more special-purpose processing devices such as an application specific integrated circuit (ASIC), a field programmable gate array (FPGA), a digital signal processor (DSP), reduced instruction set computing (RISC) microprocessor, and the like. The memory may include computer-readable media, such as synchronous dynamic random access memory (DRAM), read-only memory (ROM)), a static memory (e.g., flash memory and a data storage device). In some embodiments, the signal generator 1312 may include separate processing circuitry, such as for generating and/or configuring stimulation signals. In some embodiments, the processing circuity may be able to wirelessly communicate with computing devices external the oral appliances.

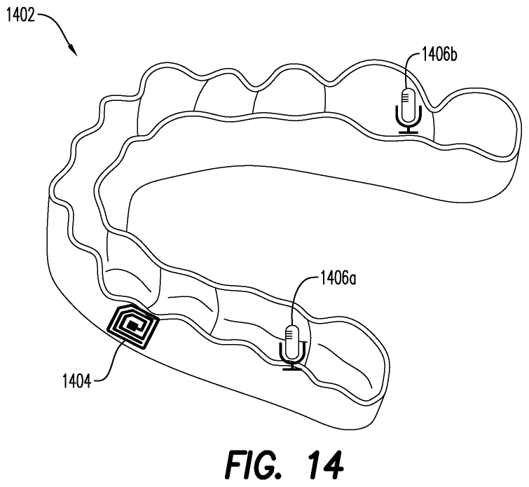

FIG. 14 illustrates a removable maxilla appliance 1402 including sound measuring devices 1406a, 1406b according to some embodiments. The maxilla appliance 1402 may be the same or similar to maxilla appliance 1306. In various embodiments, the sound measuring devices 1406a, 1406b may generate breathing data that is monitored by processing circuitry 1404 to detect patterns indicative of sleep-disordered breathing, such as inspiration or expiration levels falling below a threshold. In response to detecting a pattern indicative of sleep-disordered breathing, the processing circuitry 1404 may activate stimulation of tongue muscles. Embodiments are not limited in this context.

In some embodiments, the sound measuring devices 1406a, 1406b may include micro-electromechanical systems (MEMS) microphones. For example, the sound measuring devices 1406a, 1406b may be configured to detect upper airway collapse in instances of obstructive sleep apnea. It is contemplated that the sound measuring devices 1406a, 1406b may be configured to identify specific breathing patterns associated with sleep-disordered breathing.

There are a number of example embodiments described herein.

Example 1 is an oral appliance for a user, comprising: a body; and a stimulator including an electrode arranged on or embedded within the body so that it is in contact with a portion of a tongue of a user, wherein the stimulator is configured to stimulate a muscle of the tongue with a stimulation signal to produce contraction in the muscle of the tongue.

Example 2 is the oral appliance of Example 1 that may optionally include that the electrode is a pair of electrodes.

Example 3 is the oral appliance of Example 1 that may optionally include that the electrode includes a first electrode positioned on a left side of the tongue of the user, and a second electrode spaced apart from the first electrode and positioned on a right side of the tongue of the user.

Example 4 is the oral appliance of Example 1 that may optionally include that the electrode comprises a mesh electrode.

Example 5 is the oral appliance of Example 4 that may optionally include that the mesh electrode comprises at least one of stainless steel, gold, silver, or silver chloride.

Example 6 is the oral appliance of Example 4 that may optionally include that the mesh electrode extends about 3 centimeters along a lateral border of the tongue.

Example 7 is the oral appliance of Example 1 that may optionally include that the stimulator is configured such that the stimulation signal has a duration of between 3 and 10 seconds.

Example 8 is the oral appliance of Example 1 that may optionally include that the stimulator is configured such that the stimulation signal has an amplitude of between 0 and +/−30 volts.

Example 9 is the oral appliance of Example 1 that may optionally include that the stimulator is configured such that the stimulation signal comprises a plurality of bipolar pulses.

Example 10 is the oral appliance of Example 9 that may optionally include that the stimulator is configured such that the plurality of bipolar pulses have amplitudes that ramp-up from a first voltage to a second voltage.

Example 11 is the oral appliance of Example 9 that may optionally include that the stimulator is configured such that the plurality of bipolar pulses have amplitudes that ramp-down from a first voltage to a second voltage.

Example 12 is the oral appliance of Example 9 that may optionally include that the stimulator is configured such that the plurality of bipolar pulses include a pulse train with a ramp-up portion, a level portion, and a ramp-down portion.

Example 13 is the oral appliance of Example 1 that may optionally include a sound measuring device coupled to the body, the sound measuring device configured to detect breathing patterns of the user that are indicative of sleep-disordered breathing.

Example 14 is the oral appliance of Example 13 that may optionally include that the stimulator is configured to produce electrical impulses to stimulate the muscle of the tongue in response to detection of a breathing pattern that is indicative of a sleep-disordered breathing event.

Example 15 is the oral appliance of Example 1 that may optionally include that the electrode is in contact with a ventral-lateral surface at a mid to anterior portion of a genioglossus muscle of the tongue of the user; and the stimulator is configured to stimulate the genioglossus muscle to produce contraction in the genioglossus muscle.

Example 16 is the oral appliance of Example 1 that may optionally include that the muscle of the tongue includes at least one of a super longitudinal, inferior longitudinal, palatoglossus, styloglossus, and genioglossus muscle.

Example 17 is an oral appliance comprising: a transducer configured to generate data based on one or more physiological parameters; processing circuitry configured to detect a pattern in the data that is indicative of sleep-disordered breathing; and a stimulator configured to generate a stimulation signal including a plurality of bipolar pulses in response to detection of the pattern in the data.

Example 18 is the oral appliance of Example 17 that may optionally include that the transducer comprises a sound measuring device and the physiological parameters include an inspiration or an expiration level.

Example 19 is the oral appliance of Example 17 that may optionally include a mesh electrode configured to deliver the stimulation signal to at least a portion of a tongue muscle.

Example 20 is the oral appliance of Example 17 that may optionally include that the stimulator is configured such that the plurality of bipolar pulses have amplitudes that ramp-up from a first voltage to a second voltage.

Example 21 is a computer-implemented method comprising: monitoring breathing data of a user, the breathing data generated by a transducer; detecting a pattern in the breathing data that indicates sleep-disordered breathing; and generating a stimulation signal to stimulate a muscle of a tongue of the user in response to detection of the pattern.

Example 22 is the oral appliance of Example 21 that may optionally include that the breathing pattern comprises at least one of an inspiration level or an expiration level falling below a threshold.

This disclosure, in various embodiments, configurations and aspects, includes components, methods, processes, systems, and/or apparatuses as depicted and described herein, including various embodiments, sub-combinations, and subsets thereof. This disclosure contemplates, in various embodiments, configurations and aspects, the actual or optional use or inclusion of, e.g., components or processes as may be well-known or understood in the art and consistent with this disclosure though not depicted and/or described herein.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Approximating language, as used herein throughout the specification and claims, may be applied to modify any quantitative representation that could permissibly vary without resulting in a change in the basic function to which it is related. Accordingly, a value modified by a term such as “about” or “approximately” is not to be limited to the precise value specified. Such approximating language may refer to the specific value and/or may include a range of values that may have the same impact or effect as understood by persons of ordinary skill in the art field. For example, approximating language may include a range of +/−10%, +/−5%, or +/−3%. The term “substantially” as used herein is used in the common way understood by persons of skill in the art field with regard to patents, and may in some instances function as approximating language. In some instances, the approximating language may correspond to the precision of an instrument for measuring the value.

In this specification and the claims that follow, reference will be made to a number of terms that have the following meanings. The terms “a” (or “an”) and “the” refer to one or more of that entity, thereby including plural referents unless the context clearly dictates otherwise. As such, the terms “a” (or “an”), “one or more” and “at least one” can be used interchangeably herein. Furthermore, references to “one embodiment”, “some embodiments”, “an embodiment” and the like are not intended to be interpreted as excluding the existence of additional embodiments that also incorporate the recited features. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. In some instances, the approximating language may correspond to the precision of an instrument for measuring the value. Terms such as “first,” “second,” “upper,” “lower” etc. are used to identify one element from another, and unless otherwise specified are not meant to refer to a particular order or number of elements.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable, or suitable. For example, in some circumstances an event or capacity can be expected, while in other circumstances the event or capacity cannot occur-this distinction is captured by the terms “may” and “may be.”

As used in the claims, the word “comprises” and its grammatical variants logically also subtend and include phrases of varying and differing extent such as for example, but not limited thereto, “consisting essentially of” and “consisting of.” Where necessary, ranges have been supplied, and those ranges are inclusive of all sub-ranges therebetween. It is to be expected that the appended claims should cover variations in the ranges except where this disclosure makes clear the use of a particular range in certain embodiments.

The terms “determine”, “calculate” and “compute,” and variations thereof, as used herein, are used interchangeably and include any type of methodology, process, mathematical operation or technique.

This disclosure is presented for purposes of illustration and description. This disclosure is not limited to the form or forms disclosed herein. In the Detailed Description of this disclosure, for example, various features of some exemplary embodiments are grouped together to representatively describe those and other contemplated embodiments, configurations, and aspects, to the extent that including in this disclosure a description of every potential embodiment, variant, and combination of features is not feasible. Thus, the features of the disclosed embodiments, configurations, and aspects may be combined in alternate embodiments, configurations, and aspects not expressly discussed above. For example, the features recited in the following claims lie in less than all features of a single disclosed embodiment, configuration, or aspect. Thus, the following claims are hereby incorporated into this Detailed Description, with each claim standing on its own as a separate embodiment of this disclosure.

Advances in science and technology may provide variations that are not necessarily express in the terminology of this disclosure although the claims would not necessarily exclude these variations.

What is claimed is:

1. An oral appliance for a user, comprising:
a body; and
a stimulator including an electrode arranged on or embedded within the body so that it is in contact with a portion of a tongue of a user, wherein the stimulator is configured to stimulate a muscle of the tongue with a stimulation signal to produce contraction in the muscle of the tongue.

2. The oral appliance of claim 1, wherein:
the electrode is in contact with a ventral-lateral surface at a mid to anterior portion of a genioglossus muscle of the tongue of the user; and
the stimulator is configured to stimulate the genioglossus muscle.

3. The oral appliance of claim 1, wherein the electrode includes a first electrode positioned on a left side of the tongue of the user, and a second electrode spaced apart from the first electrode and positioned on a right side of the tongue of the user.

4. The oral appliance of claim 1, wherein the electrode comprises a mesh electrode.

5. The oral appliance of claim 4, wherein the mesh electrode comprises at least one of stainless steel, gold, silver, or silver chloride.

6. The oral appliance of claim 4, wherein the mesh electrode extends about 3 centimeters along a lateral border of the tongue.

7. The oral appliance of claim 1, wherein the stimulator is configured such that the stimulation signal has a duration of between 3 and 10 seconds.

8. The oral appliance of claim 1, wherein the stimulator is configured such that the stimulation signal has an amplitude of between 0 and +/−30 volts.

9. The oral appliance of claim 1, wherein the stimulator is configured such that the stimulation signal comprises a plurality of bipolar pulses.

10. The oral appliance of claim 9, wherein the stimulator is configured such that the plurality of bipolar pulses have amplitudes that ramp-up from a first voltage to a second voltage.

11. The oral appliance of claim 9, wherein the stimulator is configured such that the plurality of bipolar pulses have amplitudes that ramp-down from a first voltage to a second voltage.

12. The oral appliance of claim 9, wherein the stimulator is configured such that the plurality of bipolar pulses include a pulse train with a ramp-up portion, a level portion, and a ramp-down portion.

13. The oral appliance of claim 1, further comprising a sound measuring device coupled to the body, the sound measuring device configured to detect breathing patterns of the user that are indicative of sleep-disordered breathing.

14. The oral appliance of claim 13, wherein the stimulator is configured to produce electrical impulses to stimulate the muscle of the tongue in response to detection of a breathing pattern that is indicative of a sleep-disordered breathing event.

15. An oral appliance comprising:
a transducer configured to generate data based on one or more physiological parameters;
processing circuitry configured to detect a pattern in the data that is indicative of sleep-disordered breathing; and
a stimulator configured to generate a stimulation signal including a plurality of bipolar pulses in response to detection of the pattern in the data.

16. The oral appliance of claim 15, wherein the transducer comprises a sound measuring device and the physiological parameters include an inspiration or an expiration level.

17. The oral appliance of claim 15, further comprising a mesh electrode configured to deliver the stimulation signal to at least a portion of a tongue muscle.

18. The oral appliance of claim 15, wherein the stimulator is configured such that the plurality of bipolar pulses have amplitudes that ramp-up from a first voltage to a second voltage.

19. A computer-implemented method, comprising:
monitoring breathing data of a user, the breathing data generated by a transducer;
detecting a pattern in the breathing data that indicates sleep-disordered breathing; and
generating a stimulation signal to stimulate a muscle of a tongue of the user in response to detection of the pattern.

20. The computer-implemented method of claim 19, wherein the breathing pattern comprises at least one of an inspiration level or an expiration level falling below a threshold.
